(19) United States
(12) Patent Application Publication
(10) Pub. No.: US 2025/0121324 A1
(43) Pub. Date: Apr. 17, 2025

(54) AIR PURIFIER USING WATERING

(71) Applicants: Young Il LEE, Seoul (KR); Seung Chul LEE, Busan (KR); Chan Hye YUN, Uijeongbu-si (KR)

(72) Inventors: Young Il LEE, Seoul (KR); Seung Chul LEE, Busan (KR); Chan Hye YUN, Uijeongbu-si (KR)

(21) Appl. No.: 18/485,115

(22) Filed: Oct. 11, 2023

(51) Int. Cl. B01D 53/75; A61L 9/14; A61L 9/20; A62B 7/10; A62B 9/00; A62B 9/04; B01D 46/00; B01D 46/44; B01D 53/34; B01D 53/38; B01D 53/78; B01D 53/86

(52) U.S. Cl. CPC B01D 53/75; A61L 9/145; A61L 9/20; A62B 7/10; A62B 9/003; A62B 9/04; B01D 46/0028; B01D 46/442; B01D 53/346; B01D 53/38; B01D 53/78; B01D 53/8678; B01D 53/8696; A61L 2209/111; A61L 2209/12; A61L 2209/14; B01D 2258/06; B01D 2259/4541; B01D 2279/65

(57) ABSTRACT

An air purifier. A dry filter section includes a filter member, a dust sensor, and a UV lamp to primarily collect ultrafine dust particles. A suction pump is disposed to forcibly supply the intaken air with the dust particles removed. First and second wet purification sections are filled with water to secondarily purify the intaken air and purified air. A control section controls the operation of the suction pump and respective valves connecting the dry filter section, and the first and second wet purification sections. First and second bubble removers are rotatably provided in the first and second wet purification sections, respectively, to remove air-bubbles injected into the first and second wet purification sections, under the control of the control section.

### BACKGROUND

### Field

The present disclosure relates to an air purifier using watering, and more particularly, to an air purifier passing atmospheric air through a multistage filter to remove fine dust from the air, and passing the dust-free air through chemical-added water several times to provide purified air, thereby automatically providing purified air in a closed space, such as a respiratory system, a vehicle, a building, etc., and maintaining comfortable breathing conditions according to a decrease in oxygen concentration, providing excellent economic efficiency due to having a simple structure, and providing ensured safety and extended service life due to fewer failures, while purifying polluted air and pollutant gases when using various air conditioning facilities as well as electric appliances such as air conditioners, fans, and cleaners.

### Description

In general, an anionic air purifier is a device electrostatically charging fine particles in the air by applying a high voltage to a discharge electrode with positive (+) or negative (−) polarity and grounding the opposite polarity to a dust-collecting electrode trapping the charged dust. In the present disclosure, when a discharge occurs between a grounded discharge electrode with a positive (+) voltage and a dust-collecting electrode configured to generate a negative (−) high voltage, various dust particles charged with positive (+) polarity from the discharge electrode may stick to the dust-collecting electrode with negative (−) polarity, generating a large amount of anions to remove positive ions and ozone harmful to the human body, while collecting dust particles.

Therefore, a large amount of dust may accumulate on the dust-collecting electrode with a negative polarity, and a considerable amount of dust, less than that present on the dust-collecting electrode, may stick to the discharge electrode with a positive polarity, so periodic cleaning of the discharge electrode and the dust-collecting electrode is required to prevent a deterioration in the performance of the air purifier.

In cities in China, the concentration of fine or ultrafine particles is usually very high, especially in winter due to low precipitation, and sometimes the concentration of fine or ultrafine particles is unmeasurable due to the operation of heating equipment.

However, people are increasingly realizing that even as the concentration of air pollution continues to increase, they are forced to live in an environment in which it is uncomfortable to breathe, and sometimes even fall deeper and deeper into a disease without realizing it.

In addition, although the concentration of air pollution in other parts of the world is not as high as in China, the concentration of fine dust or ultrafine dust is getting higher and higher, and it is becoming more and more frequent that the concentration of fine dust or ultrafine dust is slightly uncomfortable to breathe at times.

Even a small excess of pure moisture in the air is not good for the health of the lungs, but people are now breathing in a lot of moisture particles contained in smog, as well as fine particles harmful to the human body, so it is urgently required to take measures to directly remove fine particles floating and traveling in the air for the health of people.

Countries around the world have realized the serious impact of rapidly increasing air pollution on the global environment and signed the “2015 Paris Climate Agreement.” However, because the Agreement is not fully enforceable, countries may be lukewarm in terms of compliance with the agreement for their own industrial competitiveness.

If China, which prides itself as being the center of the world, passively responds to the unenforceable “2015 Paris Climate Agreement,” the concentration of air pollution is bound to increase, so that cities in China could become like imaginary futuristic cities in which the air is extremely polluted, since peoples in the cities are increasingly unable to see the sky and sun due to thick smog, even during the daytime, and in the future, despite experiencing no rain at all, the humidity in the air may be very high, so many particles of moisture contained in the smog may become water droplets and continue to flow down on the walls of buildings.

Countries around the world are now recognizing that even though their own air pollution is not as bad as it could be, China's example illustrates the need to tighten emission standards for all polluting facilities. However, they are not taking seriously the fact that global air quality will never improve unless the great amount of fine particles, suspended in the air and moving around, is actively and directly removed.

Nevertheless, the worse the air pollution, the less competitive goods can be produced, and the greater the health risks to the public, the Chinese government complacently temporarily shuts down all production facilities and suspends public transportation whenever there is a major international event occurring in China.

However, Chinese government officials have so far been unable to come up with any effective measures to directly remove the fine particulate matter from the air, which may be very embarrassing.

As a G2 country, China is obliged to comply with the “2015 Paris Climate Agreement” in an exemplary manner, and is realizing the need to actively improve all pollution sources that generate fine dust, but with astronomical costs and a significant decrease in national competitiveness, the Chinese government is feeling very burdened and unable to provide a solution.

However, even if the country now starts taking measures to improve air quality, people will have to live in a severely polluted environment for decades to come, before the country's air quality is improved to some extent.

Therefore, as a technology developed to prevent the adverse effects of fine dust (including ultrafine dust) on the human body, an air purifier of Patent Document 1 is known, manufactured, and marketed.

However, with the structure of the air purifier, there is a problem that the discharge efficiency is significantly reduced due to the inconvenience in separating and cleaning the electrodes, as well as poor cleaning of the electrodes.

An air purifier disclosed in Patent Document 3 was developed in consideration of the above situation. The air purifier of Patent Document 3 includes: a rectangular cylindrical main body having a power switch and a belt hook on a rear side thereof; a battery casing provided at a lower end of the main body; a filter unit sequentially mounted in the main body; a fan having a fan drive motor mounted on one side of the main body; a lid covering the main body; a flexible hose connected to an exhaust hole formed in one side of the lid; and a helmet or mask connected to the flexible hose. The filter unit includes a deodorizing filter, an antibacterial filter, and a bactericidal filter. A mesh-type air inlet is formed on one side of the lid, while a flat filter is mounted on the surface of the air inlet. An air exhaust hole is formed in the other side of the lid through which the purified air is discharged. A spiral groove and a ball insertion hole are formed in an inner surface of the end side of the air outlet. Elastic balls are formed on both ends of the flexible hose. A pipe for connecting the flexible hose to the helmet or mask is formed to have a long slot with a ball insertion hole in an inner surface of the pipe. This configuration allows the air purifier to be miniaturized, making it easy to carry. It is possible to connect the mask or helmet to the air purifier so that motorcycle drivers can inhale fresh air purified by the air purifier through the mask or helmet when riding a motorcycle. It is also possible to reduce the production costs by making the structure simple and straightforward, thereby reducing the price of the product.

However, since the air purifier of Patent Document 3 requires the helmet or mask to inhale purified fresh air, the air purifier entails the disadvantage that a general person who is not a motorcycle driver must wear the helmet or mask unnecessarily, and the price of the product increases due to the unnecessary helmet or mask.

In addition, a portable air purifier disclosed in Patent Document 4 includes: a casing, a cover part, an air purification part, a motor part, a control module part, a battery, a power terminal part, an ionizer having a through-hole, a moisture removal part containing a dehumidifying agent embedded in a through-hole, and a speaker. The casing is a spherical-shaped member including first and second casing portions with a compartment formed therein. The first casing portion has a hemispherical shape with an inlet, through which external air or contaminated air to be purified is introduced, formed at one-side lower end and with the power terminal part disposed at the bottom surface so as to be connected with the battery to supply external power; the second casing portion has an outlet, through which purified air is discharged, formed at one-side upper end. The cover part is an arc-shaped member disposed between the first casing and the second casing so as to rotate about a rotary axis. The cover part forms an engagement protrusion at one end to engage with an engagement groove of the casing through interference-fit engagement. The air purification section includes a filter portion, a filter casing, and a handle. The filter portion is wound around a central through-hole thereof. The filter casing has a through-hole into which a portion of the battery is fixedly inserted. The motor part is fastened in the second casing through the motor casing. The power terminal part is connected to an external power supply through an electrode. With this configuration, the air purifier is easy to move and carry, and can perform various functions such as air purification, dehumidification, and sound output to improve user's satisfaction.

However, the air purifier of Patent Document 4 has problems in that due to the complex structure thereof, not only is the product expensive, but also there is a risk of failure, it is difficult to use by an individual alone because there is no structure such as a hose connecting to a respirator that can be used by the individual alone, and it is inconvenient to use because the power supply must be provided separately along with a power cable.

In order to solve these problems, Patent Document 5 describes an air purifier using watering to purify contaminated air by passing the contaminated air through water. The air purifier of Patent Document 5 includes: a chemical container disposed on the ceiling of first and second underwater purification sections to inject, into the water in the first and second underwater purification sections, the chemicals for sterilization, disinfection, and removing harmful substances in gases or smokes; and a blower bursting air bubbles on the water surface in the first and second underwater purification sections. A cartridge filter part is a cuboidal housing having four sides, with at least three sides of the four sides being respectively configured as multiple filters. A dust sensor is mounted on the remaining side to measure a concentration of fine dust. Each of the multiple filters is composed of a photocatalytic filter, a carbon filter, and a HEPA filter configured to operate in the concentration of the discharged oxygen ranging 21% or less. However, the air purifier of Patent Document 5 has problems in that the air purification performance thereof is relatively low and improvement of performance is required.

The information disclosed in the Background section is only provided for a better understanding of the background and should not be taken as an acknowledgment or any form of suggestion that this information forms prior art that would already be known to a person having ordinary skill in the art.

### RELATED ART DOCUMENT

Patent Document 1: KR 10-2001-0017510 A
Patent Document 2: KR 20-0178327 Y1
Patent Document 3: KR 10-2011-0097019 A
Patent Document 4: KR 10-2017-0138289 A
Patent Document 5: KR 10-2097902 B1

### BRIEF SUMMARY

Various aspects of the present disclosure provide an air purifier with a simple, straightforward structure and performance improvements at any size by passing atmospheric air through a multistage filter to remove fine dust from the air, and passing the dust-free air through chemical-added water several times to provide purified air, thereby automatically providing purified air in a closed space, such as a respiratory system, a vehicle, a building, etc., and maintaining comfortable breathing conditions according to a decrease in oxygen concentration, providing excellent economic efficiency due to having a simple structure, providing ensured safety and extended service life due to fewer failures, and purifying polluted air and pollutant gases when using various air conditioning facilities as well as electric appliances such as air conditioners, fans, and cleaners.

According to an aspect, an air purifier may include: a dry filter section including a filter member configured to filter dust particles such as fine dust in the intaken air, a dust sensor configured to measure the concentration of dust particles in the intaken air after removing the dust particles therefrom, and a UV lamp configured to sterilize the intaken air after removing the dust particles therefrom, so as to primarily collect ultrafine dust particles; a suction pump disposed inside the dry filter section to forcibly supply the intaken air after removing the dust particles therefrom; a first wet purification section filled with a predetermined level of water to secondarily purify the intaken air supplied from the suction pump in a wet purification process including air-bubble injection; a second wet purification section disposed adjacently to the first wet purification section with a predetermined level of water filled therein to secondarily purify the purified air supplied from the first wet purification section in a wet purification process including air-bubble injection; a control section configured to control the operation of the suction pump and respective valves connecting the dry filter section, the first wet purification section, and the second wet purification section; and first and second bubble removers rotatably provided at the first and second wet purification sections, respectively, to remove air-bubbles injected into the first and second wet purification sections, under the control of the control section. A user may be able to breathe purified air during outdoor activities by wearing the air purifier, to breathe purified air in a vehicle by mounting the air purifier on the vehicle, and to breathe purified air in a building by disposing the air purifier in the building. The filter member applied to the dry filter section may be one of a photocatalytic filter, a carbon filter, and a HEPA filter, or combinations thereof.

A first magnetic coupler may be provided between the dry filter section and the first wet purification section to fix portions mutually disposed therebetween.

A second magnetic coupler may be provided between the first wet purification section and the second wet purification section to fix portions mutually disposed therebetween.

Each of the first and second bubble removers may include: a rotary shaft rotatably disposed vertically; a drive motor provided at one end of the rotary shaft to operatively rotate the rotary shaft; a bubble removal blade provided on one side of the rotary shaft to rotate with the rotary shaft to remove bubbles in the water; and a circulation blade provided on another side of the rotary shaft to rotate with the rotary shaft to circulate the purified air over the water.

The bubble removal blade may include a plurality of radially arranged mesh nets having microscopic holes.

The first and second wet purification sections may be provided with first and second chemical containers, respectively, to provide a purification solution, harmless to the human body, to the water accommodated in the first and second wet purification sections.

By providing the configuration as set forth above, the present disclosure has the advantages of automatically providing purified air in a closed space, such as a respiratory system, a vehicle, a building, etc., and maintaining comfortable breathing conditions according to a decrease in oxygen concentration, providing excellent economic efficiency due to the simple structure, providing ensured safety and extended service life due to fewer failures, and purifying polluted air and pollutant gases when using various air conditioning facilities as well as electric appliances such as air conditioners, fans, and cleaners.

### DESCRIPTION OF DRAWING

The above and other objectives, features, and advantages of the present disclosure will be more clearly understood from the following detailed description, taken in conjunction with the accompanying drawing, in which:

FIG. 1 is a configuration diagram illustrating an air purifier using watering according to an embodiment of the present disclosure.

### DETAILED DESCRIPTION

In the following description of the present disclosure, detailed descriptions of known functions and components incorporated herein will be omitted to avoid rendering the subject matter of the present disclosure unclear. Terms used herein are defined in light of the functions of the present disclosure, which may change depending on user, operator's intention, or common practice. Definitions should therefore be taken in the context of the entire specification. The terms used in the detailed description are intended only to describe embodiments of the present disclosure and should not be construed as being limiting. Unless expressly used otherwise, expressions in the singular form include the meaning of the plural form. In this description, expressions such as “including” or “comprising” are intended to refer to certain features, numbers, steps, actions, elements, parts or combinations thereof, and should not be construed to exclude the existence or possibility of one or more other features, numbers, steps, actions, elements, parts or combinations thereof other than those described.

In each of the systems illustrated in the drawing, elements in some instances may have the same or different reference numerals to suggest that the elements shown may be different or similar. However, elements may have different implementations and may work with any or all of the systems shown or described herein. The various elements illustrated in the drawing may be the same or different. Which one is referred to as the first element or the second element is arbitrary.

As used herein, references to a component “transmitting,” “delivering,” or “providing” data or signals to another component include transmitting data or signals from one component directly to another component, as well as transmitting data or signals to another component through at least one other component.

Hereinafter, an exemplary embodiment will be described in detail with reference to the accompanying drawing so that a person having ordinary knowledge of the present disclosure belonging to the same technical field can easily practice the exemplary embodiment.

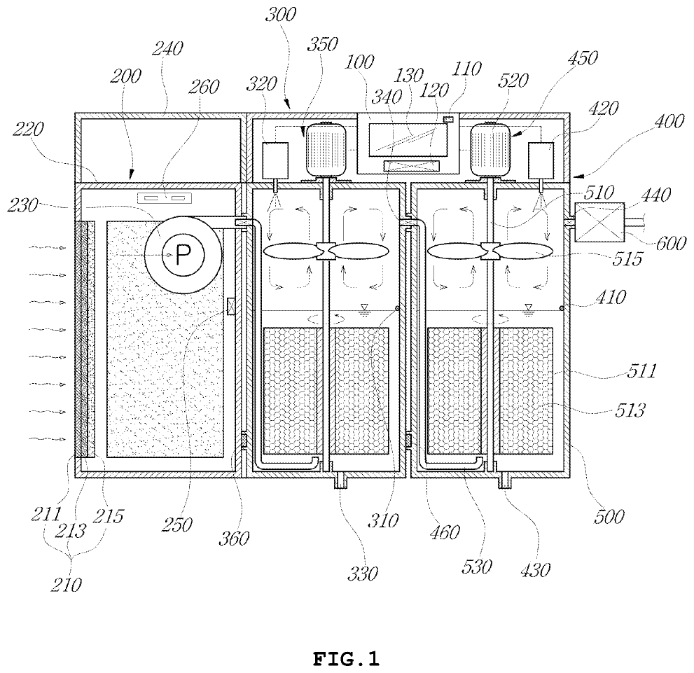

As illustrated in FIG. 1, according to the present disclosure, an air purifier using watering essentially includes a dry filter section 200, a suction pump 230, a first wet purification section 300, a second wet purification section 400, a control section 100, and first and second bubble removers 350 and 450. The dry filter section 200 is configured to primarily collect ultrafine dust particles, and includes a filter member 210 configured to filter dust particles such as fine dust in the intaken air, a dust sensor configured to measure the concentration of dust particles in the intaken air after removing the dust particles therefrom, and a UV lamp 260 configured to sterilize the intaken air after removing the dust particles therefrom. The suction pump 230 is disposed inside the dry filter section 200 to forcibly supply the intaken air after removing the dust particles therefrom. The first wet purification section 300 is filled with a predetermined level of water to secondarily purify the intaken air supplied from the suction pump 230 in a wet purification process including air-bubble injection. The second wet purification section 400 is disposed adjacently to the first wet purification section 300 with a predetermined level of water filled therein to secondarily purify the purified air supplied from the first wet purification section 300 in a wet purification process including air-bubble injection. The control section 100 is configured to control the operations of the suction pump 230 and respective valves connecting the dry filter section 200, the first wet purification section 300, and the second wet purification section 400. The first and second bubble removers 350 and 450 are rotatably provided in the first and second wet purification sections 300 and 400, respectively, to remove air-bubbles injected into the first and second wet purification sections 300 and 400, under the control of the control section 100.

The first wet purification section 300 also includes: a first water level sensor 310 indicating the level of the filled water, a battery 120 provided therein, an oxygen supply disposed next to the battery 120, an inlet pipe connected through the dry filter section 200, a suction pump 230 and a check valve connected to the end of the inlet pipe in water, nozzles, and drains.

On the other hand, a dehumidifying filter section 600 is preferably further connected to the end of the second wet purification section 400 to which a second air outlet 330 is connected. The dehumidifying filter section 600 is configured to discharge the final dried purified air by capturing and removing moisture contained in the purified air as the purified air passes through the first wet purification section 300 and the second wet purification section 400.

Here, the oxygen supplies may be provided to the first wet purification section 300 and the second wet purification section 400, respectively, according to a signal from the control section 100 connected to an oxygen sensor.

The second wet purification section 400 may have a second water level sensor 410 indicating the level of the filled water; and an inlet pipe, an exhaust pipe, and a drain pipe formed at an upper portion of the first wet purification section 300 and a lower portion of the second wet purification section 400, respectively.

Therefore, the air purifier may allow the user to breathe purified air during outdoor activities by wearing the air purifier, to breathe purified air in a vehicle by mounting the air purifier on the vehicle, to breathe purified air in a building by installing the air purifier in the building, and may purify contaminated air and gases when applied to electric appliances such as air conditioners, fans, and cleaners.

A filter element 210 applied to the dry filter section 200 may include any one or a combination of a photocatalytic filter 211, a carbon filter 213, and a HEPA filter 215.

The dry filter section 200 may include a filter frame 220, a cuboidal housing with filter elements 210 on at least three sides.

A dust sensor 250 that measures the concentration of fine or ultrafine dust is disposed on one side of the interior of the filter frame, and each of the filter members 210 is composed of a photocatalytic filter 211, a carbon filter 213, and a HEPA filter 215, which can be separated and removed from the air purifier as needed, and the air may be purified with only the first wet purification section 300 and the second wet purification section 400.

The dry filter section 200 may include a filter frame 220, and the first wet purification section 300 and the second wet purification section 400 may be detachably formed into a purification frame 500.

The purification frame 500 may be centrally equipped with an oxygen sensor to provide a signal through a display 130 indicating the oxygen concentration of the purified air.

On the other hand, a first magnetic coupler 360 may be provided between the dry filter section 200 and the first wet purification section 300 to detachably fix portions mutually disposed therebetween.

A second magnetic coupler 460 may be provided between the first wet purification section 300 and the second wet purification section 400 to detachably fix portions mutually disposed therebetween.

The first wet purification section 300 and the second wet purification section 400 may be arbitrarily disposed in two or more stages depending on the amount of fine dust.

For example, the wet purification section may be disposed in three or more stages in a location in which a large amount of fine dust is generated, such as a thermal power plant or an incinerator.

The first and second bubble removers 350 and 450 each may include: a rotary shaft 510 rotatably disposed vertically; a drive motor 520 provided at one end of the rotary shaft 510 to operatively rotate the rotary shaft 510; a bubble removal blade 511 provided on one side of the rotary shaft 510 to rotate with the rotary shaft 510 to remove bubbles in the water; and a circulation blade 515 provided on another side of the rotary shaft 510 to rotate with the rotary shaft 510 to circulate the purified air over the water.

The first bubble remover 350 and the second bubble remover 450 include the rotary shaft 510, the circulation blade 515, and the removal blade 511, and the rotary shaft 510 is telescopically extended like an antenna and the removal blade 511 rotates on the water surface to remove a plurality of air bubbles so that the purified air is well discharged from the water surface.

Here, a blower fan may be disposed instead of or in addition to the circulation blade 515 of the first and second bubble removers 350 and 450.

It is also desirable that the circulation blades 515 and the removal blades 511 of the first and second bubble removers 350 and 450 be operated separately as needed.

The removal blade 511 may include a plurality of radially arranged mesh nets 513 having microscopic holes.

Here, the mesh nets 513 may be continuously arranged in multiple layers in the longitudinal direction of the rotary shaft 510, or may be radially arranged around the rotary shaft 510.

The first wet purification section 300 and the second wet purification section 400 may be equipped with a first chemical container 320 and a second chemical container 420, respectively, which provide a purification solution, harmless to the human body, to the water accommodated in the first wet purification section 300 and the second wet purification section 400.

When the air purifier using watering according to the present disclosure is used outdoors, the user fills the first wet purification section 300 and the second wet purification section 400 with water to a level of the first water level sensor 310 and the second water level sensor 410, inserts a nosepiece connected to a waist-worn air intake means into the nostrils, and turns on a power switch 110 on the control section 100.

Then, the suction pump 230 in the first wet purification section 300 operates, and air with fine and ultrafine particles removed through the filter member 210 enters the dry filter section 200, passes through the water in the first wet purification section 300 to the top, and then passes through the water in the second wet purification section 400 to the top.

Here, a very small hole or a nozzle may be disposed at the end of the inlet pipe in the water to bubble-inject the air into the first wet purification section 300 to not only efficiently purify a large amount of air, but also supply oxygen in the purified air to the water in the form of dissolved oxygen.

On the other hand, the suction pump 230 and the nozzle may also be disposed at a portion where external air is introduced into the first wet purification section 300 through a check valve to blow air into the first wet purification section 300 to efficiently purify fine dust contained in the air.

The air purified through the first wet purification section 300 is then dispersed into the water of the second wet purification section 400 through the inlet pipe, purified through the water of the second wet purification section 400, and then discharged to the top of the second wet purification section 400, so that the purified air with fine dust and bacteria removed therefrom may be supplied to a user to breathe through the air discharge hose of the air intake means and through the nosepiece.

At this time, the concentration of oxygen present in the air in the upper part of the second wet purification section 400 is measured by an oxygen sensor in the oxygen supply, and if the measured oxygen concentration (OC) is below the concentration preset in the control section 100, the first bubble remover 350, the second bubble remover 450, and the blower fan are operated, and the shaft lengths of the first bubble remover 350 and the second bubble remover 450 are adjusted so that the removal blades 511 of the first bubble remover 350 and the second bubble remover 450 are immerged in the water.

On the other hand, in an enclosed space such as an apartment, office, laboratory, etc., the oxygen sensor is operated when the oxygen concentration in the enclosed space decreases, and if the oxygen concentration in the enclosed space is lower than 21%, the window is opened and the air purifier using watering according to the present disclosure is operated. Then, the air entering the air purifier through the window is purified and supplied to the user by the air purifier according to the present disclosure as described above.

If the oxygen concentration in a vehicle is also lower than 21%, the vehicle window is opened to allow external air to enter the vehicle, and the air purifier according to the present disclosure is activated. Then, the air entering the air purifier according to the present disclosure through the window is purified and supplied to the user by the multipurpose air purifier according to the present disclosure, and the purified air is provided to the user, so that users of the air purifier, especially children, if they know how to use the air purifier, will not suffocate even if they are trapped in the vehicle.

In addition, when all the windows of the vehicle are closed, the present air purifier is automatically activated in conjunction with the closing of the vehicle's entrance door, thereby principally preventing the accident of suffocation of children trapped in a vehicle.

In addition, the present air purifier may be disposed in a specific place in a public facility where people reside, such as in the roof of a large apartment building, to supply purified air to each household through air pipes in the supply manner of an oxygenator in a hospital.

In particular, when a fire is detected by a fire detector in the event of a fire, the control section 100 interlocked therewith blocks the air from entering the first wet purification section 300 through the dry filter section 200 by the backflow prevention by the check valve, opens the check valve to introduce air from outside the fire area into the first wet purification section 300 and purify the air, and supplies the purified air to the required place of each household through the air pipe 530, thereby dramatically avoiding suffocation, which is a common cause of injury or death due to smoke from a fire.

In addition, in the operation of the present air purifier, chemicals that sterilize, disinfect, and remove harmful substances in gases or smokes are periodically injected from the first chemical container 320 and the second chemical container 420.

In addition, in case the present air purifier is disposed in a vehicle, if all windows and doors of the vehicle are closed, the present air purifier automatically starts to operate in conjunction with the closing of the windows and doors, thereby preventing accidents in which children are trapped in the vehicle and suffocate.

By providing the configuration as set forth above, the present disclosure has the advantages of automatically providing purified air in a closed space, such as a respiratory system, a vehicle, a building, etc., and maintaining comfortable breathing conditions according to a decrease in oxygen concentration, providing excellent economic efficiency due to the simple structure, and ensured safety and extended service life due to fewer failures, and purifying polluted air and pollutant gases when using various air conditioning facilities as well as electric appliances such as air conditioners, fans, and cleaners.

The terms and words used in this specification and claims described above are not to be construed in their ordinary or dictionary sense, but should be interpreted in a sense and concept consistent with the technical idea of the invention, based on the principle that the inventor may properly define the concept of a term to best describe his/her invention.

Therefore, it is to be understood that the configurations illustrated in the drawing and embodiments described herein are merely one most exemplary embodiment of the invention and are not exhaustive of the technical ideas of the invention so that there may be various equivalents and variations which may be substituted for them at the time of filing.

What is claimed is:

1. An air purifier comprising:
a dry filter section including a filter member configured to filter dust particles such as fine dust in the intaken air, a dust sensor configured to measure the concentration of dust particles in the intaken air after removing the dust particles therefrom, and a UV lamp configured to sterilize the intaken air after removing the dust particles therefrom, so as to primarily collect ultrafine dust particles;
a suction pump disposed inside the dry filter section to forcibly supply the intaken air after removing the dust particles therefrom;
a first wet purification section filled with a predetermined level of water to secondarily purify the intaken air supplied from the suction pump in a wet purification process including air-bubble injection;
a second wet purification section disposed adjacently to the first wet purification section with a predetermined level of water filled therein to secondarily purify the purified air supplied from the first wet purification section in a wet purification process including air-bubble injection;
a control section configured to control the operation of the suction pump and respective valves connecting the dry filter section, the first wet purification section, and the second wet purification section; and
first and second bubble removers rotatably provided at the first and second wet purification sections, respectively, to remove air-bubbles injected into the first and second wet purification sections, under the control of the control section,
wherein a user is able to breathe purified air during outdoor activities by wearing the air purifier and to breathe purified air in a vehicle by mounting the air purifier on the vehicle, and the air purifier is able to purify polluted air and pollutant gases when various air conditioning facilities as well as electric appliances such as air conditioners, fans, and cleaners are used,
wherein the filter member applied to the dry filter section comprises any one or combinations of a photocatalytic filter, a carbon filter, and a HEPA filter.

2. The air purifier of claim 1, wherein a first magnetic coupler is provided between the dry filter section and the first wet purification section to fix portions mutually disposed therebetween.

3. The air purifier of claim 1, wherein a second magnetic coupler is provided between the first wet purification section and the second wet purification section to fix portions mutually disposed therebetween.

4. The air purifier of claim 1, wherein each of the first and second bubble removers comprises:
a rotary shaft rotatably disposed vertically;
a drive motor provided at one end of the rotary shaft to operatively rotate the rotary shaft;
a bubble removal blade provided on one side of the rotary shaft to rotate with the rotary shaft to remove bubbles in the water; and
a circulation blade provided on another side of the rotary shaft to rotate with the rotary shaft to circulate the purified air over the water.

5. The air purifier of claim 4, wherein the bubble removal blade comprises a plurality of radially arranged mesh nets having microscopic holes.

6. The air purifier of claim 1, wherein the first and second wet purification sections are provided with first and second chemical containers, respectively, to provide a purification solution, harmless to the human body, to the water accommodated in the first and second wet purification sections.

7. The air purifier of claim 1, wherein a second air outlet of the second wet purification section is further connected with a dehumidifying filter section to discharge purified dried air by capturing and removing moisture contained in the air purified while passing through the first wet purification section and the second wet purification section.
